(19) United States
(12) Patent Application Publication
(10) Pub. No.: US 2009/0221863 A1
(43) Pub. Date: Sep. 3, 2009

(54) HF akylation process

(75) Inventors: Ramon A. Strauss, Herndon, VA (US); Brian A. Cunningham, The Colonnade (SG); Jeffrey M. Fitt, Centreville, VA (US); Ramesh R. Hemrajani, Millington, NJ (US)

Correspondence Address: ExxonMobil Research & Engineering Company, P.O. Box 900, 1545 Route 22 East, Annandale, NJ, 08801-0900, US

(73) Assignee: ExxonMobil Research and Engineering Comapny, Annandale, NJ (US)

(21) Appl. No.: 12/320,400

(22) Filed: Jan. 26, 2009

Related U.S. Application Data
(63) Continuation-in-part of application No. 11/987,428, filed on Nov. 30, 2007.
(60) Provisional application No. 60/873,940, filed on Dec. 11, 2006.

(51) Int. Cl. C07C 2/60; B01J 14/00

(52) U.S. Cl. 585/710; 422/187

(57) ABSTRACT

An HF alkylation process for producing gasoline boiling range alkylate product by the alkylation of a light olefin with an isoparaffin in the presence of hydrogen fluoride as an alkylation catalyst, in which the content of organic fluorides in the alkylation product stream is reduced by recontact with additional HF alkylation acid after which the hydrocarbon phase and the denser acid-containing phase are separated in a hydrocyclone. The use of the hydrocyclone enables the acid inventory of the unit to be significantly reduced which still achieving satisfactory removal of the unwanted fluoride by-products.

### CROSS REFERENCE TO RELATED APPLICATION

This application is a continuation-in-part of application Ser. No. 11/987,428, filed 30 Nov. 2007, which claimed priority from Application Ser. No. 60/873,940, filed 11 Dec. 2006.

### FIELD OF THE INVENTION

This invention relates to isoparaffin/olefin alkylation and more particularly, to hydrofluoric acid (HF) alkylation. In this specification, the term “alkylation” will be used to refer to the isoparaffin/olefin alkylation process used to make gasoline blend components useful in aviation and motor gasolines and “HF alkylation” to this process using hydrofluoric acid as the catalyst.

### BACKGROUND OF THE INVENTION

The isoparaffin/olefin alkylation process is widely used to manufacture a high octane quality blend component for aviation and motor gasoline which is also valued for its relatively low vapor pressure, low sensitivity and, because of its freedom from aromatic components, its environmental acceptability. The process typically reacts a C₃ to C₅ olefin with isobutane in the presence of an acidic catalyst to produce the alkylate product.

Industrial alkylation processes have historically used concentrated hydrofluoric (HF) or sulfuric acid catalysts under relatively low temperature conditions. Acid strength is preferably maintained at 88 to 94 weight percent by the continuous addition of fresh acid and the continuous withdrawal of spent acid.

Hydrofluoric and sulfuric acid alkylation processes share inherent drawbacks including environmental and safety concerns, acid consumption, and sludge disposal but in spite of efforts to develop an inherently safe alkylation process, both processes have achieved widespread utilization with the HF process being noted for producing a higher quality product with more favorable unit economics. Although hydrogen fluoride, or hydrofluoric acid (HF) is highly toxic and corrosive, extensive experience in its use in the refinery have shown that it can be handled safely, provided the hazards are recognized and precautions taken. The HF alkylation process is described in general terms in Modern Petroleum Technology, Hobson et al (Ed), Applied Science Publishers Ltd. 1973, ISBN 085334 487 6.

One problem encountered during the operation of the HF alkylation unit is that organic fluorides are produced as by-products of the reactions which take place. These organic fluorides are produced in relatively minor amounts, typically up to 1000 ppm in the alkylate product, but are nevertheless undesirable as they have to be removed as contaminants and, moreover, the formation of fluorides affects HF consumption as well as producing waste materials which require disposal if they cannot be consumed in the course of the process itself. This problem has been noted in the basic HF process as well as in the modified HF alkylation process using a vapor suppressant. U.S. Pat. No. 3,204,011 (Hettick et al), for example, refers to the production of these waste materials as does U.S. Pat. No. 6,114,593 (Randolph et al) which describes the modified HF alkylation process using a sulfone vapor suppressant e.g. an organic sulfone such as sulfolane. A number of prior proposals for removing these by-products are described in U.S. Pat. No. 3,204,011 (Hettick et al) as well as a proposal to the same end of treating the alkylate reaction product containing the organic fluorides with a mixture of HF and isobutane so that the isobutane is alkylated by the alkyl fluoride with the concomitant production of HF. U.S. Pat. No. 3,910,771 (Chapman) describes a riser contactor device intended to convert alkyl fluorides in the HF alkylate into alkylate by contact with the isoparaffin and additional HF acid. Randolph, in U.S. Pat. No. 6,114,593 makes a similar proposal for the modified HF process using an eductor to mix HF with the initial alkylate reaction product from a first stage settler, followed by allowing the mixture to settle in a second stage settler from which the acid is returned to the eductor. U.S. Pat. No. 5,185,487 (Love et al) also discloses the use of an eductor to mix organic fluoride-containing alkylate with HF acid to release the HF and make more alkylate.

Although the use of recontactors to reduce the level of by-product fluorides is generally effective, it does have the disadvantage of increasing the acid inventory of the unit. This is not only undesirable economically but also from the viewpoint of safety since in the event of an uncontrolled release of the HF acid a larger volume of acid is present to be released. Accordingly, it would be desirable to effect of recontacting method which enables the initial alkylate product containing the undesired organic fluorides to be brought into contact with the HF acid to regenerate acid and convert the fluorides to additional alkylate but, at the same time, to do this without increasing the HF inventory to the extent required by conventional recontactor techniques. We have now developed a method of this kind.

### SUMMARY OF THE INVENTION

In the process according to the present invention, the alkylate product from the acid settler containing the organic fluoride by-products is mixed in a recontactor with additional HF acid to produce an effluent stream of reduced organic fluoride content containing a hydrocarbon phase and an acid phase. This stream is then separated into a hydrocarbon phase and an acid phase in a hydrocyclone after which the acid can be returned to the reactor and to the recontactor. Because the phase separation is carried out in the hydrocyclone, the internal volume of a recontactor settler can, if used at all, be significantly reduced with a consequential reduction in the unit HF inventory.

According to the present invention, therefore, an HF alkylation process for producing gasoline boiling range alkylate product by the alkylation of light olefins with an isoparaffin in the presence of hydrogen fluoride as an alkylation catalyst, reacts the isoparaffin with the olefin in the presence of HF to produce an effluent stream comprising alkylate product as a hydrocarbon phase, organic fluoride by-product and a denser acid phase. The hydrocarbon phase of the effluent stream either on its own or still with the acid phase, is then mixed with additional HF acid to convert the organic fluorides after which the hydrocarbon phase is separated from the denser acid phase in a hydrocyclone.

Generally, the initial reaction between the isoparaffin and the olefin will be carried out in a conventional reactor with an initial phase separation taking place in a primary settler from which the hydrocarbon phase can be withdrawn for recontacting with the additional acid to remove the organic fluorides. The hydrocarbon phase may then be separated and worked up, for example, in the conventional manner by removal of unreacted isoparaffin in the isostripper (de-isobutanizer) and other requisite processing. Following the recontacting and separation in the hydrocyclone a further phase separation may, if desired, be brought about in a secondary settler. This settler, however, can be relatively small in size compared to the settlers conventionally used after recontactors in conventional units since the major part of the phase separation can be effected in the hydrocyclone. By the use of efficient recontactor design, moreover, utilizing jet mixing with internal recycle, as described below, a further reduction in acid inventory relative to the size of the unit may be achieved.

### DRAWINGS

In the accompanying drawings:

FIG. 1 is a simplified process schematic of a conventional HF alkylation unit with recontactor;

FIG. 2 is a simplified process schematic of one form of an HF alkylation unit with recontactor and hydrocyclone phase separator;

FIG. 3 is a simplified process schematic of an alternative form of an HF alkylation unit with recontactor, hydrocyclone phase separator and a secondary or recontactor settler;

FIG. 4 is a simplified section of a recontactor with jet mixing and internal recycle, and

FIG. 5 is a schematic of a suitable hydrocyclone configuration.

### DETAILED DESCRIPTION

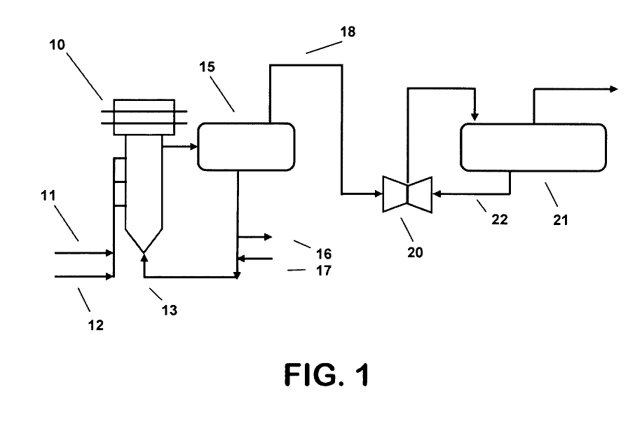

FIG. 1 shows a conventional HF alkylation unit with recontactor. The olefin and isoparaffin feeds are introduced into reactor 10 through lines 11 and 12 respectively with HF alkylation acid being brought in by means of line 13. The reactor may be of conventional baffled upflow type with the hydrocarbons introduced at multiple levels through spargers extending through the sidewalls of the reactor and HF acid introduced at the bottom. The alkylate product may be withdrawn at the top of the reaction volume and cooling may be provided by a cooling head at the top of the reactor. A typical reactor of this type is shown, for example, in U.S. Pat. No. 3,914,111 (Anderson). The reactor effluent then passes to a primary settler 15 in which a phase separation between the less dense hydrocarbon phase and the denser acid phase takes place. Acid is withdrawn from the lower part of the settler and recycled to the reactor with provision of lines 16, 17 for the withdrawal of a slipstream of acid and the addition of fresh make-up acid to maintain constant acid inventory.

The hydrocarbon phase is withdrawn from the upper portion of the primary settler and passes through line 18 to eductor/mixer 20 in which it is mixed with additional HF acid drawn from the lower portion of secondary settler 21 passing by way of line 22. Eductor/mixer 20 causes intense mixing between the hydrocarbon phase and the additional HF acid with the hydrocarbon being dispersed in fine droplets to ensure good contact with the added acid. The contact of the hydrocarbon phase containing the organic fluoride by-products with the additional acid is effective to convert the fluorides to additional alkylate by alkylation of residual isoparaffin the effluent stream with the fluoride by-products. At the same time, HF acid is regenerated by release of the fluorine from the fluorides. If desired, additional isoparaffin may be added through line 18 prior to eductor/mixer 20 to ensure that adequate isoparaffin is present for the alkylation reaction in the recontacting step; this, however, will normally not be necessary since the alkylation reaction is normally carried out in the presence of excess isoparaffin and the relative amount of the organic fluorides is quite low. The two phases are separated in secondary settler 21 with the acid phase from this settler passing to the eductor/mixer 20. Conventionally, lines for the addition of fresh acid and acid withdrawal are provided (not shown) and this acid circuit may be integrated with the primary acid circuit to the reactor.

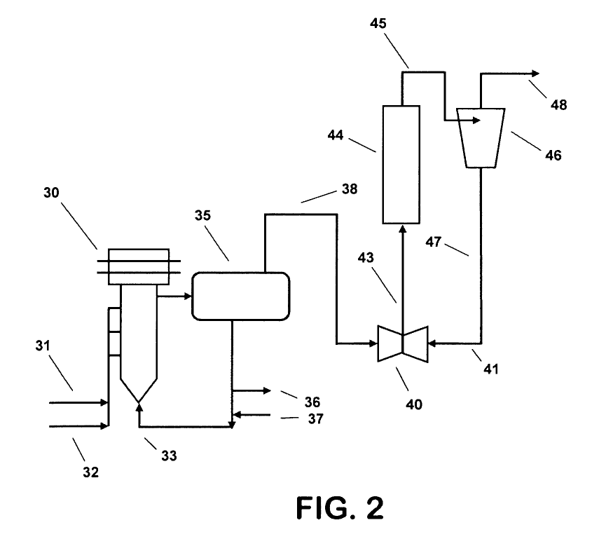

FIG. 2 is a simplified process schematic for an HF unit using the combination of a recontacting section with a hydrocyclone to effect phase separation of the acid phase and the hydrocarbon phase. The initial reactor/settler arrangement is similar to that shown in FIG. 1, with olefin and isoparaffin feeds entering reactor 30 through lines 31, 32 and acid HF recirculated from settler 35 through line 33 fitted with acid withdrawal and make-up lines 36, 37. From settler 35, the hydrocarbon phase is taken by way of line 38 to the recontacting section which includes an external eductor/mixer 40 in which the hydrocarbon phase is intimately mixed with additional acid introduced from acid recycle line 41. In this case, however, the hydrocarbon/acid mix from the eductor/mixer travels through line 43 to microcontactor 44 in which the contact between the hydrocarbon phase containing the organic fluorides and the acid phase is promoted by vigorous mixing under high shear conditions. From microcontactor 44, the mixture proceeds by way of line 45 to hydrocyclone 46 in which the hydrocarbon phase, now with its content of organic fluoride reduced significantly by reaction with the added acid, is separated from the acid phase which passes out through the lower axial outlet of the hydrocyclone to acid recycle line 41. The hydrocarbon phase, largely free of organic fluoride by-product, issues from the vortex outlet of hydrocyclone 46 and is taken through line 48 to the product recovery section of the alkylation unit.

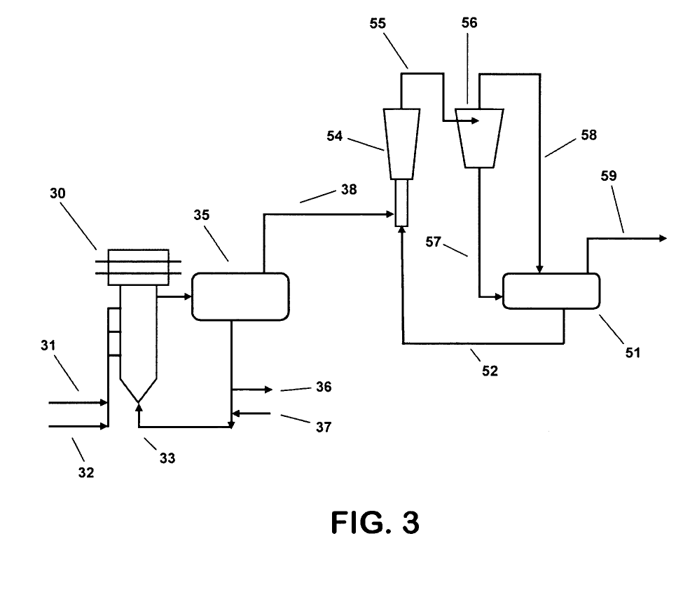

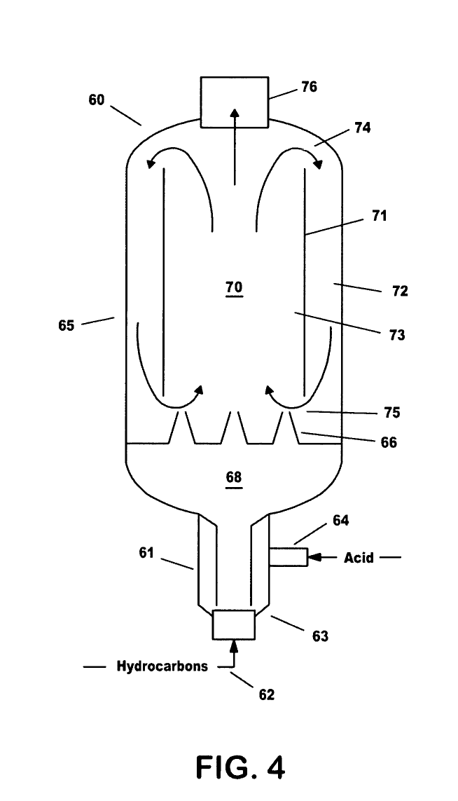

The alkylation unit shown in FIG. 3 is a modification of the one shown in FIG. 2; in this case, the recontacting section does not have an external eductor/mixer as shown in FIG. 2 but, rather, an internal eductor integrated with the microcontactor. Accordingly, the primary reactor circuit is numbered with the same numerals. From primary settler 35 the hydrocarbon phase passing through line 38 is taken directly to the inlet of integrated eductor/microcontactor 54 into which added HF acid is introduced from line 52, taking its acid supply from the lower region of a secondary settler 51 of relatively small volume. Recontacting of the hydrocarbon phase and the additional acid to reduce the organic fluoride content takes place in microcontactor 54 after which the mixed hydrocarbon and acid phases pass through line 55 to the volute of hydrocyclone 56 in which phase separation takes place. The denser acid phase passes out of the hydrocyclone through the lower axial outlet and then through line 57 to the lower region of a secondary or recontactor mini-settler 51, which may be provided to supplement the separation in the hydrocyclone by permitting any hydrocarbon in the acid stream to settle out by gravity. The hydrocarbon product with its reduced level of organic fluorides passes out of the upper axial outlet of hydrocyclone 56 and is taken to the product recovery section through line 58. The hydrocarbons separated from the acid in the mini-settler can be removed by way of line 59 and taken to product recovery in line 58 together with the hydrocarbons from the hydrocyclone. Because the secondary mini-settler is provided merely to remove any hydrocarbon passing out with the acid stream, its volume can be made quite small so that the acid inventory of the unit is not significantly increased beyond the level in its absence. Again, as with units of the type shown in FIG. 1 without the secondary mini-settler, the volume of acid in the unit may be significantly reduced since separation of the immiscible acid and hydrocarbon phases following the recontacting is effected in the hydrocyclone. If sufficiently vigorous mixing can be achieved in the eductor and the following transfer line, it may be possible to eliminate the microcontactor as a separate or integrated element. In this case, the effluent from the eductor is taken directly to the hydrocyclone along a transfer line with a length adequate for the recontact to take place to effect removal of the fluorides. If, however, more extended recontact is found necessary, a microcontactor may be used, for example, as shown in FIG. 4.

As may be perceived from the above description, the present invention departs from the prior art techniques in its use of an enhanced separation technique for the recontact mixture of hydrocarbons and added acid. Instead of relying solely on a settler to effect the separation, the hydrocyclone rapidly brings about a effective phase separation which can them be supplemented by a further separation in the secondary mini-settler if required, e.g. because of occasional operational constraints or unit upsets. In addition, the use of a high shear contactor will enhance mixing and so afford potential for shorter recontact times. A form of the microcontactor with an integrated eductor/mixer, as used in the unit of FIG. 3, is shown in FIG. 4. This contactor is generally in the form of a vertically disposed cylindrical vessel 60 with a lower eductor/mixer section 61. The hydrocarbon phase from the primary settler enters at axial inlet 62 and passes to the venturi section indicated in outline at 63. The additional acid to be used for the recontact step enters through radial inlet 64 on the side of the eductor section so that the passage of the hydrocarbon through the venture will draw in the acid. Mixing of the acid and hydrocarbon phases is then initiated in this section before the mixture passes upwards through the lower part of the vessel into the enlarged contact section 65. Contact section 65 has a lower inlet chamber 68 fitted with jet outlets 66 (three are illustrated although in actual practice, the number would be substantially greater). The mixture of acid and hydrocarbon passes through the jet outlets into the upper contact section 70 after undergoing vigorous mixing and dispersal of the phases under the high shear conditions encountered in the jet outlets.

The upper contact section 70 has an annular baffle 71 which extends most of the axial length of the section defining an annular flow path 72 between the baffle and the inner walls of the contact section and a central flow path 73 for the ascending fluid mixture. The central flow path has an annular overflow 74 at the top and a similarly dimensioned annular recirculation passage 75 at the bottom in the region of the jet outlets. This construction promotes extremely efficient mixing with a very short residence time in the contactor: the mixture entering the upper contact section exits the jet outlets at high speed and creates its own eductor effect to draw mixture from the annular region 72 between baffle 71 and the inner wall of the upper section into the ascending flow of fluid by way of recirculation passage 75. The central axial outlet 76 of the contactor permits a controlled flow of mixture to leave the contact section after a determined average residence time with recirculation of the mixture passing upwards through central flowpath 73 taking place over the overflow 74, down recirculation flow path 72 and radially inwards through recirculation passage 75 back into the ascending flow of mixed fluid.

FIG. 4 illustrates the principles upon which a high shear, short contact time, low volume contactor may be constructed. The length/diameter ratio of the contactor may be varied according to the desired product quality and residence time which is itself dependent on the content of fluoride by-product in the effluent from the primary settler. This, in turn, is dependent on the conditions used in the initial alkylation including feed identity and ratio, temperature and presence of any vapor suppressant additives such as sulfolane. The dimensioning of the flow passages is dependent on the desired recirculation rate relative to the overall flow rate.

The basic requirement for the hydrocyclone is that it effects a substantial separation of the two liquid phases. Given this objective the conventional construction utilizing a volute inlet for the hydrocarbon/acid mixture with a lower axial outlet for the denser phase (acid) and an upper axial outlet for the less dense phase (hydrocarbon) will be the general choice. In the hydrocyclone, the mixed eductor effluent enters the cylindrical chamber using the tangential or volute inlet nozzle which drives the fluid to rotate. The induced centrifugal forces accelerate the denser acid phase towards the wall in the underflow direction and this phase then exits through the apex orifice. The less dense phase moves towards the center as a reversed vortex and it leaves the hydrocyclone through the vortex finder and the overflow outlet. The general objective of this separation step is to produce an overflow consisting of hydrocarbon phase with less than 2% acid. Various types of hydrocyclone conforming to this prescription may be used with variations such as the type with a bell-shaped axial vortex finder tube and central core, for example, as shown in U.S. Pat. No. 6,024,874 (Lott). Other hydrocyclone designs useful for effecting separation of oil/water mixtures are shown, for example, in U.S. Pat. No. 4,237,006 (Colman), 4,576,724 (Colman), 4,764,287 (Colman), U.S. Pat. No. 4,749,490 (Smyth) and U.S. Pat. No. 5,017,288 (Thew).

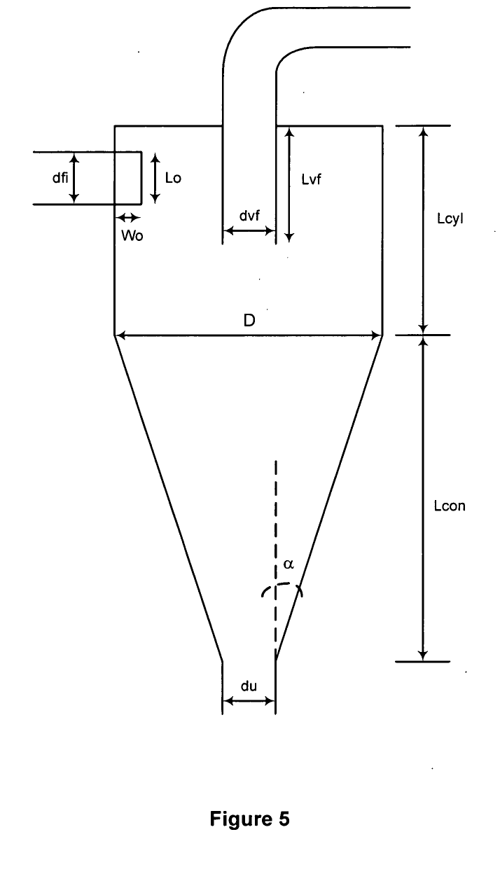

A suitable hydrocyclone configuration for separating the oil phase from the aqueous acid phase is shown in FIG. 5. The hydrocyclone is of generally conventional overall configuration with a tangential volute inlet, an axial underflow outlet for the denser acid phase and an opposed overflow outlet for the less dense oil phase. The hydrocyclone is characterized by the parameters given in Table 1 below, with reference to FIG. 5.

TABLE 1
Hydrocyclone design parameters

| | Parameter | Design |
|---|---|---|
| D | Inside diameter, mm. | D |
| dfi | Feed inlet diameter, mm. | D/3.5 |
| Lo | Inlet nozzle height, mm. | 0.8 dfi |
| Wo | Inlet nozzle width, mm. | Lo/2 |
| dvf | Vortex finder diameter, mm. | 0.35 D |
| Lvf | Vortex finder length, mm. | 0.6 D |
| du | Underflow diameter, mm. | 0.225 D |
| α | Conical section angle | 5° (1-20°) |
| Lcyl | Cylindrical section length, mm. | D |
| Lcon | Conical section length, mm. | (D − du)/2 tan α |

If required, a plurality of serially connected hydrocyclones may be used to achieve the desired degree of separation between the oil and acid phases. In such cases, the overflow outlet of the first hydrocyclone will be connected by a transfer line of suitable size to the volute inlet of the second hydrocyclone and so on.

As noted above, the process may be operated with a vapor suppressant additive to improve safety margins in the event of an uncontrolled acid release from the unit. The vapor suppressant additives normally contemplated are those which reduce the volatility of the HF acid. Compounds of this type which have been proposed include organic sulfones, ammonia, amines such as the lower alkylamines (methyl to pentyl), pyridine, alkylpyridines, picoline, melamine, hexamethylenetetramine. A number of different sulfones have been proposed for this purpose but the one generally preferred is sulfolane (tetramethylenesulfone) with 3-methylsulfolane and 2,4-dimethylsulfolane also being suitable. A more detailed description of vapor suppressant additives of this type is given in U.S. Pat. No. 6,114,593 to which reference is made for this description. When a vapor suppressant additive is used the process is often referred to as modified HF alkylation (MHF).

As conventional with HF alkylation (including MHF), the light olefins used will normally be C₃-C₄ olefins with preference being given to butylenes although propylene and ethylene may also be used with corresponding changes in product properties and in the appropriate process conditions, as is known. The isoparaffin most favored is isobutane. Conditions will, apart from the use of the recontactor and its associated hydrocyclone, be conventional for the process.

1. An HF alkylation process for producing gasoline boiling range alkylate product by the alkylation of a light olefin reactant with an isoparaffin reactant in the presence of hydrogen fluoride as an alkylation catalyst, which process comprises:
reacting the isoparaffin with the olefin in the presence of an HF acid alkylation catalyst to produce, as an effluent stream, (i) a hydrocarbon phase comprising alkylate product and organic fluoride by-product and (ii) a denser acid phase; recontacting the hydrocarbon phase of the effluent stream with additional HF alkylation acid to remove the organic fluorides and form a hydrocarbon phase and a denser acid-containing phase and separating the hydrocarbon phase from the denser acid phase in a hydrocyclone.

2. A process according to claim 1 in which the isoparaffin and the olefin are reacted to form the effluent stream which is subjected to an initial separation in a primary settler from which the hydrocarbon phase can be withdrawn for recontacting with the additional HF alkylation acid to remove the organic fluorides.

3. A process according to claim 2 in which, following the recontacting and separation in the hydrocyclone the denser acid phase is passed to a secondary settler for a further phase separation step.

4. A process according to claim 1 in which the hydrocarbon phase from the primary settler is recontacted with the additional HF alkylation acid by jet mixing in a recontactor.

5. A process according to claim 4 in which the hydrocarbon phase from the primary settler is recontacted with the additional acid by jet mixing in a recontactor with internal recycle in the recontactor of the mixed hydrocarbon phase and added HF alkylation acid.

6. A process according to claim 5 in which the recontactor comprises a recontacting vessel comprising a cylindrical vessel with a lower eductor/mixer section having inlets for the hydrocarbon phase and for the additional HF alkylation acid, the inlet for the hydrocarbon phase being connected to a hydrocarbon outlet of the primary settler and the inlet for the additional acid being connected to an acid outlet of the hydrocyclone.

7. A process according to claim 6 in which the recontactor comprises a lower inlet chamber communicating with the lower eductor/mixer section with jet mixing outlets communicating with a downstream upper contact section having an axially extensive annular baffle defining an annular flow path between the baffle and the inner walls of the contact section and a central flow path for fluid mixture ascending from the jet outlets.

8. A process according to claim 7 in which the central flow path defined by the baffle has an annular overflow at its upper end and an annular recirculation passage at its lower end in the region of the jet outlets.

9. A process according to claim 4 in which the hydrocarbon phase from the primary settler is recontacted with the additional acid by jet mixing in an eductor/mixer from which the mixed hydrocarbon and acid phases are passed directly to the inlet of the hydrocyclone.

10. A process according to claim 1 in which the HF alkylation acid comprises a vapor suppressant additive.

11. A process according to claim 9 in which the vapor suppressant additive comprises sulfolane.

12. An HF alkylation unit for producing gasoline boiling range alkylate product by the alkylation of a light olefin reactant with an isoparaffin reactant in the presence of hydrogen fluoride as an alkylation catalyst, which unit comprises:
a reactor for reacting the isoparaffin with the olefin in the presence of HF alkylation acid to produce an effluent stream at an outlet of the reactor comprising (i) a hydrocarbon phase comprising alkylate product and organic fluoride by-product and (ii) a denser acid phase;
a primary settler having an inlet connected to the reactor outlet, a lower acid outlet and an upper hydrocarbon outlet;
a recontacting section for mixing hydrocarbon with additional HF alkylation acid, the recontacting section having a hydrocarbon inlet connected to the upper outlet of the primary settler, an inlet connected to a source of additional HF alkylation acid and an outlet;
a hydrocyclone for separating a hydrocarbon phase from a denser acid phase, the hydrocyclone having an inlet connected to the outlet of the recontacting section.

13. A unit according to claim 12 in which the hydrocyclone has an outlet for the denser acid phase connected to an inlet of a secondary settler.

14. A unit according to claim 12 in which the recontacting section includes an eductor/mixer having an inlet connected to the upper outlet of the primary settler and an inlet connected to a source of additional HF alkylation acid.

15. A unit according to claim 14 in which the recontacting section comprises an eductor/mixer having an inlet connected to the upper outlet of the primary settler, an inlet for the source of additional HF alkylation acid and an outlet for mixed hydrocarbon/acid phases connected by means of a transfer line to the inlet of the hydrocyclone.

16. A unit according to claim 12 in which the recontacting section includes a recontacting vessel comprising a cylindrical vessel with a lower eductor/mixer section having inlets for the hydrocarbon phase and for the additional acid, the inlet for the hydrocarbon phase being connected to a hydrocarbon outlet of the primary settler and the inlet for the additional acid being connected to an acid outlet of the hydrocyclone.

17. A unit according to claim 16 in which the recontactor comprises a lower inlet chamber communicating with the lower eductor/mixer section, the lower inlet chamber having jet mixing outlets communicating with a downstream upper contact section having an axially extensive annular baffle defining an annular flow path between the baffle and the inner walls of the contact section and a central flow path for fluid mixture ascending from the jet outlets.

18. A unit according to claim 17 in which the central flow path defined by the baffle has an annular overflow at its upper end and an annular recirculation passage at its lower end in the region of the jet outlets.
